(19) United States
(12) Patent Application Publication
(10) Pub. No.: US 2024/0307695 A1
(43) Pub. Date: Sep. 19, 2024

(54) GUIDEWIRE FOR PACING DURING REPLACEMENT HEART VALVE DELIVERY

(71) Applicant: BOSTON SCIENTIFIC SCIMED, INC., MAPLE GROVE, MN (US)

(72) Inventor: Spencer Fodness-Bondhus, Columbia Heights, MN (US)

(73) Assignee: BOSTON SCIENTIFIC SCIMED, INC., MAPLE GROVE, MN (US)

(21) Appl. No.: 18/598,313

(22) Filed: Mar. 7, 2024

Related U.S. Application Data
(60) Provisional application No. 63/452,554, filed on Mar. 16, 2023.

(51) Int. Cl. A61N 1/362; A61F 2/24; A61M 25/09; A61N 1/05

(52) U.S. Cl. CPC A61N 1/3629; A61F 2/2427; A61N 1/056; A61M 2025/09091; A61M 2025/09183

(57) ABSTRACT

A guidewire may be used for delivering and implanting a replacement heart valve implant and may also be used for pacing the patient's heart during implantation of the replacement heart valve implant. The guidewire includes an elongate shaft including a distal section and a proximal section extending proximally from the distal section, a coiled portion disposed within the distal section, and one or more electrodes disposed within the coiled portion, the one or more electrodes adapted for pacing the heart. The proximal section is adapted to provide an electrical connection with a pacing system.

### CROSS REFERENCE TO RELATED APPLICATIONS

This application claims the benefit of priority of U.S. Provisional Application No. 63/452,554 filed Mar. 16, 2023, the entire disclosure of which is hereby incorporated by reference.

### TECHNICAL FIELD

The present disclosure pertains to medical devices and methods for using medical devices. More particularly, the present disclosure pertains to a guidewire for delivering and implanting a replacement heart valve implant with concurrent pacing.

### BACKGROUND

A wide variety of intracorporeal medical devices have been developed for medical use, for example, intravascular use. Some of these devices include guidewires, catheters, medical device delivery systems (e.g., for stents, grafts, replacement valves, etc.), and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example may be found in a guidewire for delivering a replacement heart valve implant. The guidewire includes an elongate shaft including a distal section and a proximal section extending proximally from the distal section, a coiled portion disposed within the distal section, and one or more electrodes disposed within the coiled portion, the one or more electrodes adapted for pacing the heart. The proximal section is adapted to provide an electrical connection with a pacing system.

Alternatively or additionally, the elongate shaft may include a polymeric coating disposed on the coiled portion.

Alternatively or additionally, the one or more electrodes disposed within the coiled portion may include spots in the coiled portion in which the polymeric coating has been removed to expose metal underneath the polymeric coating.

Alternatively or additionally, at least some of the one or more electrodes may be adapted to contact a ventricular wall of a heart when the coiled portion is disposed within a ventricle of the heart.

Alternatively or additionally, the proximal section of the elongate shaft may include a bare metal portion that is adapted to accommodate an alligator clip connecting the elongate shaft with the pacing system.

Alternatively or additionally, the proximal section of the elongate shaft may include a bare metal proximal end that is adapted to accommodate an electrical connection with the pacing system.

Alternatively or additionally, the elongate shaft may include a polymeric coating over an entirety of the elongate shaft with the exception of the one or more electrodes and a bare metal region of the proximal section.

Alternatively or additionally, at least a proximal-most region of the proximal section may include an uninsulated metal core and an insulated coil extending over the uninsulated metal core.

Alternatively or additionally, the coiled portion is curved in a first direction in a first plane, the elongate shaft includes a reverse curve portion curved in a second direction opposite the first direction, and the reverse curve portion is disposed proximal of the coiled portion.

Alternatively or additionally, the reverse curve portion may be curved in the second direction within the first plane.

Another example may be found in a guidewire adapted to deliver pacing pulses. The guidewire includes an elongate shaft including a distal section and a proximal section extending proximally from the distal section, the proximal section including an electrical contact segment adapted to make electrical contact with a pacing system. The elongate shaft is adapted for delivering a replacement heart valve implant, the elongate shaft including a coiled portion disposed within the distal section, the coiled portion curved in a first direction in a first plane, and wherein the elongate shaft includes a reverse curve portion curved in a second direction opposite the first direction. The elongate shaft includes a reverse curve portion curved in a second direction opposite the first direction, the reverse curved portion disposed proximal of the coiled portion. The coiled portion includes one or more electrodes electrically coupled with the electrical contact segment.

Alternatively or additionally, the elongate shaft may include a polymeric coating disposed on the coiled portion.

Alternatively or additionally, the one or more electrodes electrically coupled with the electrical contact segment may include spots in the coiled portion in which the polymeric coating has been removed to expose metal underneath the polymeric coating.

Alternatively or additionally, at least some of the one or more electrodes may be adapted to contact a ventricular wall of a heart when the coiled portion is disposed within a ventricle of the heart.

Alternatively or additionally, the electrical contact segment may include a bare metal portion that is adapted to accommodate an alligator clip connecting the elongate shaft with the pacing system.

Alternatively or additionally, the electrical contact segment may include a bare metal proximal end that is adapted to accommodate an electrical connection with the pacing system.

Alternatively or additionally, the elongate shaft may include a polymeric coating over an entirety of the elongate shaft with the exception of the one or more electrodes and a bare metal region of the proximal section.

Alternatively or additionally, the electrical contact segment may include an uninsulated metal core and an insulated coil extending over the uninsulated metal core.

Another example may be found in a method of treating a native heart valve of a patient's heart. The method includes advancing a catheter through the native heart valve into a ventricle of the patient's heart and advancing a guidewire out a distal end of the catheter into the ventricle of the patient's heart, wherein the guidewire includes an elongate shaft including a distal section and a proximal section extending proximally from the distal section. The elongate shaft includes a coiled portion disposed in the distal section, the coiled portion being curved in a first direction in a first plane. The elongate shaft includes a reverse curve portion curved in a second direction opposite the first direction. The reverse curve portion is disposed proximal of the coiled portion. The method includes positioning the coiled portion within the ventricle of the patient's heart such that a distal portion of the proximal section is spaced radially inward from an ostium of the native heart valve when the coiled portion of the elongate shaft is positioned within the ventricle of the patient's heart. The method includes removing the catheter while maintaining the coiled portion of the elongate shaft within the ventricle of the patient's heart. A deployment device is advanced over the guidewire to the native heart valve. The method includes rapidly pacing via the guidewire in order to temporarily interrupt native heart rhythm and deploying a replacement heart valve implant within the native heart valve, the replacement heart valve implant including an expandable framework and a plurality of valve leaflets disposed within the expandable framework. The deployment device is withdrawn without contacting the expandable framework of the replacement heart valve implant.

Alternatively or additionally, the reverse curve portion may urge the distal portion of the proximal section of the elongate shaft toward a center of the native heart valve.

The above summary of some embodiments, aspects, and/or examples is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a schematic view of an illustrative guidewire;

FIGS. 2 through 7 are schematic views showing a method of pacing while delivering a replacement heart valve implant using the illustrative guidewire of FIG. 1;

FIG. 8 is a schematic view of a portion of an illustrative guidewire;

FIG. 9 is a schematic view of a portion of an illustrative guidewire;

FIG. 10 is a schematic view of a portion of an illustrative guidewire;

FIG. 11 is a cross-sectional view taken along the line 11-11 of FIG. 10;

FIG. 12 is a cross-sectional view taken along the line 12-12 of FIG. 10;

FIG. 13 is a schematic view of a portion of an illustrative guidewire;

FIG. 14 is a schematic view of a portion of an illustrative guidewire; and

FIG. 15 is a schematic view of a portion of an illustrative guidewire.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the present disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications may be disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the present disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of a device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

The terms “transaortic valve implantation” and “transcatheter aortic valve implantation” may be used interchangeably and may each be referred to using the acronym “TAVI”. The terms “transaortic valve replacement” and “transcatheter aortic valve replacement” may be used interchangeably and may each be referred to using the acronym “TAVR”. The terms TAVI and TAVR may be used to refer to the same or similar procedures and in at least some embodiments, the terms TAVI and TAVR may be used interchangeably.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Diseases and/or medical conditions that impact the cardiovascular system are prevalent throughout the world. Traditionally, treatment of the cardiovascular system was often conducted by directly accessing the impacted part of the system. For example, treatment of a blockage in one or more of the coronary arteries was traditionally treated using coronary artery bypass surgery. As can be readily appreciated, such therapies are rather invasive to the patient and require significant recovery times and/or treatments. More recently, less invasive therapies have been developed, for example, where a blocked coronary artery could be accessed and treated via a percutaneous catheter (e.g., angioplasty). Such therapies have gained wide acceptance among patients and clinicians.

Some mammalian hearts (e.g., human, etc.) include four heart valves: a tricuspid valve, a pulmonary valve, an aortic valve, and a mitral valve. Some relatively common medical conditions may include or be the result of inefficiency, ineffectiveness, or complete failure of one or more of the valves within the heart. Treatment of defective heart valves poses other challenges in that the treatment often requires the repair or outright replacement of the defective valve. Such therapies may be highly invasive to the patient. Disclosed herein are medical devices and/or procedures that may be used within a portion of the cardiovascular system in order to diagnose, treat, and/or repair the system, for example during and/or in conjunction with a TAVI or TAVR procedure, or in place of a TAVI or TAVR procedure in patients not suitable for such. At least some of the medical devices and/or procedures disclosed herein may be delivered and/or performed percutaneously and, thus, may be much less invasive to the patient, although other surgical methods and approaches may also be used. The devices disclosed herein may also provide a number of additional desirable features and benefits as described in more detail below. For the purpose of this disclosure, the discussion below is directed toward the treatment of a native aortic valve and will be so described in the interest of brevity. This, however, is not intended to be limiting as the skilled person will recognize that the following discussion may also apply to a mitral valve or another heart valve with no or minimal changes to the structure and/or scope of the disclosure. Similarly, the medical devices and/or procedures disclosed herein may have applications and uses in other portions of a patient's anatomy, such as but not limited to, arteries, veins, and/or other body lumens.

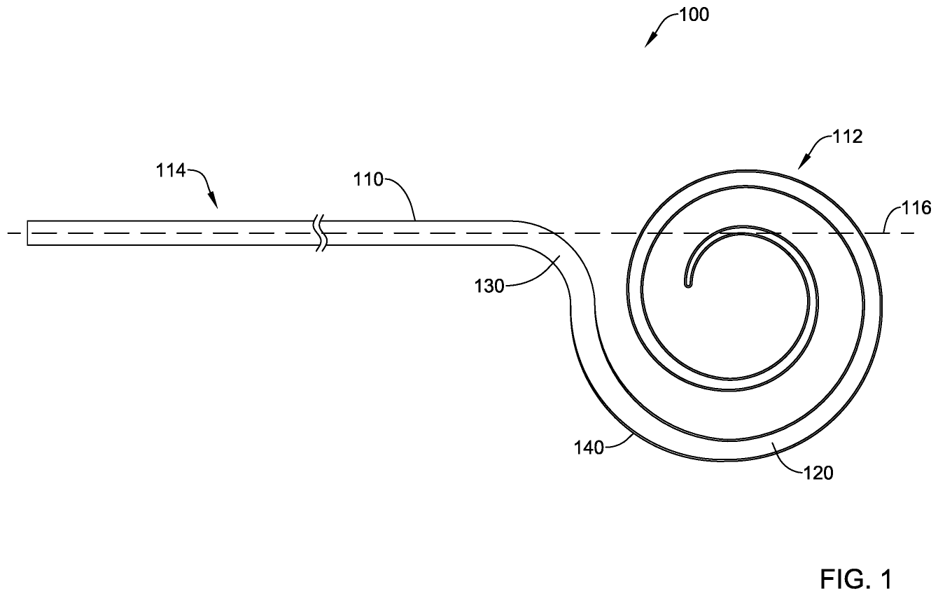

FIG. 1 is a schematic view of an illustrative guidewire 100 for delivering a replacement heart valve to a native heart valve. In some instances, the guidewire 100 may also be adapted to allow its use in delivering pacing pulses to the heart during the process of delivering the replacement heart valve. In some instances, the guidewire 100 may include an elongate shaft 110 including a distal section 112 and a proximal section 114 extending proximally from the distal section 112. In some instances, the proximal section 114 may define a central longitudinal axis 116, which may be projected distally of the proximal section 114 for reference.

The elongate shaft 110 may include a coiled portion 120 disposed within the distal section 112 in an unbiased and/or unconstrained state, wherein the coiled portion 120 of the elongate shaft 110 is curved in a first direction in a first plane, as viewed proximally to distally. In some instances, the coiled portion 120 may be curved in the first direction in the first plane in a distal direction along the elongate shaft 110. In some instances, the coiled portion 120 may extend, from proximal to distal along the length of the elongate shaft 110, in the first direction in the first plane. In some instances, the central longitudinal axis 116 may be disposed within the first plane. Other configurations are also contemplated. In some instances, the first direction may be counterclockwise. In some instances, the first direction may be clockwise.

In some instances, the elongate shaft 110 may include a reverse curve portion 130 curved in a second direction opposite the first direction in the unbiased and/or unconstrained state, as viewed proximally to distally. In some instances, the reverse curve portion 130 may be disposed within and/or may be curved in the second direction within the first plane. In some instances, the reverse curve portion 130 may be curved in the second direction in the first plane in the distal direction along the elongate shaft 110. In some instances, the reverse curve portion 130 may extend, from proximal to distal along the length of the elongate shaft 110, in the second direction in the first plane. In some instances where the first direction is counterclockwise, the second direction may be clockwise. In some instances where the first direction is clockwise, the second direction may be counterclockwise. In at least some instances, the reverse curve portion 130 may be disposed proximal of the coiled portion 120 of the elongate shaft 110.

In some instances, the reverse curve portion 130 may be at least partially disposed in the proximal section 114 of the elongate shaft 110. In some instances, the reverse curve portion 130 may extend from a distal portion of the proximal section 114 of the elongate shaft 110 into the distal section 112 of the elongate shaft 110. In some instances, the reverse curve portion 130 may span a joint and/or a transition region between the proximal section 114 of the elongate shaft 110 and the distal section 112 of the elongate shaft 110. In some instances, the reverse curve portion 130 may be disposed entirely within the proximal section 114 of the elongate shaft 110.

In some instances, the coiled portion 120 of the elongate shaft 110 may extend laterally on opposing sides of a second plane containing the central longitudinal axis 116 of the proximal section 114 of the elongate shaft 110 in the unbiased and/or unconstrained state. In some instances, a first portion of the coiled portion 120 of the elongate shaft 110 may be disposed on a first side of the second plane containing the central longitudinal axis 116 of the proximal section 114 of the elongate shaft 110, and a second portion of the coiled portion 120 of the elongate shaft 110 may be disposed on a second side of the second plane containing the central longitudinal axis 116 of the proximal section 114 of the elongate shaft 110, wherein the second side of the second plane is opposite the first side of the second plane relative to the second plane. In some instances, the second plane may be oriented at an oblique angle to the first plane. In at least some instances, the second plane may be oriented substantially perpendicular to the first plane.

In some instances, the elongate shaft 110 may have a selected level of axial stiffness and/or pushability characteristics while also having a selected level of lateral stiffness and/or flexibility to permit navigation through the patient's vasculature. In some instances, the proximal section 114 of the elongate shaft 110 may be laterally stiffer than the distal section 112 of the elongate shaft 110. In some instances, the distal section 112 may be more flexible than the proximal section 114 of the elongate shaft 110. Other configurations are also contemplated.

In some instances, the distal section 112 of the elongate shaft 110 may be tapered radially inwardly in a distal direction. For example, a proximal portion of the distal section 112 of the elongate shaft may have an outer diameter that is greater than an outer diameter of a distal portion of the distal section 112. In some instances, the distal section 112 of the elongate shaft 110 may be tapered radially inwardly from the proximal section 114 of the elongate shaft 110 to a distalmost tip of the elongate shaft 110. In some instances, the distal section 112 of the elongate shaft 110 may be tapered continuously in the distal direction. In some instances, the distal section 112 of the elongate shaft 110 may be tapered in a stepwise fashion in the distal direction. In some instances, at least a portion of the coiled portion 120 may be tapered radially inwardly in the distal direction. In some instances, an entirety of the coiled portion 120 may be tapered radially inwardly in the distal direction.

In some instances, the elongate shaft 110 may include and/or may be formed from a metallic material. In some instances, the elongate shaft 110 is formed from stainless steel. Other configurations are also contemplated. Some suitable but non-limiting examples of materials for the elongate shaft 110 are discussed below. In some instances, the elongate shaft 110 may include a polymeric coating 140 disposed on the coiled portion 120 of the elongate shaft 110 and/or the distal section 112 of the elongate shaft 110. In at least some instances, the proximal section 114 of the elongate shaft 110 may be devoid of the polymeric coating 140. Some suitable but non-limiting examples of materials for the polymeric coating are discussed below.

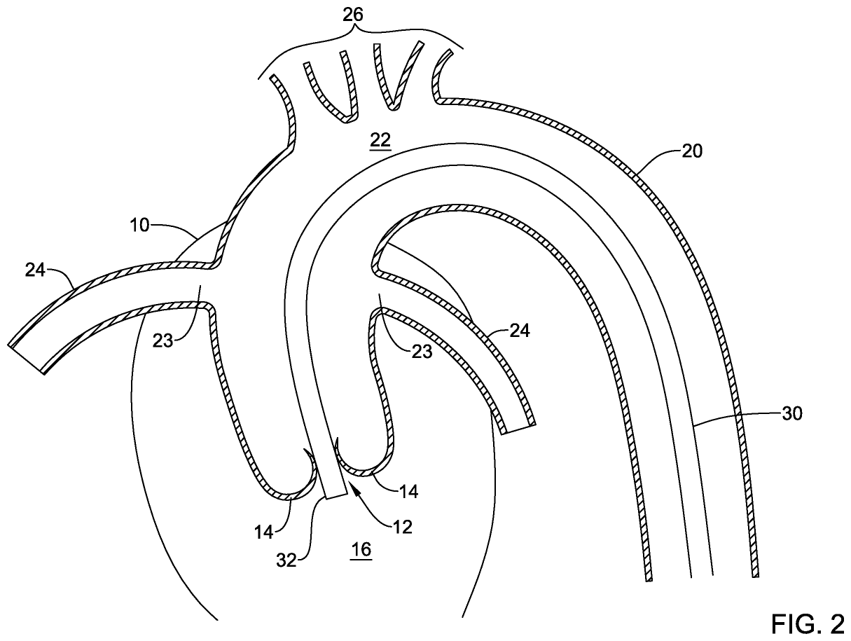

As noted, the guidewire 100 may be used for delivering and implanting a replacement cardiac valve within a patient. In some instances, the guidewire 100 may be adapted to also be able to be used for pacing the heart during the replacement valve implantation process. FIGS. 2 through 7 provide an illustrative but non-limiting example of using the guidewire 100 for delivery and implantation of a replacement cardiac valve while optionally using the guidewire 100 to deliver pacing pulses during the implantation process. FIGS. 2 through 7 include a schematic view of some of the cardiac anatomy of a patient's heart 10. The anatomy includes an aortic valve 12 having valve leaflets 14, a left ventricle 16, and certain connected vasculature, such as the aorta 20 connected to the aortic valve 12 of the patient's heart 10 by the aortic arch 22, the coronary arteries 24, the ostia 23 of the coronary arteries 24, and other large arteries 26 (e.g., subclavian arteries, carotid arteries, brachiocephalic artery) that extend from the aortic arch 22 to important internal organs. The discussion herein is directed toward use in treating the aortic valve 12 and will be so described in the interest of brevity. This, however, is not intended to be limiting as the skilled person will recognize that the following discussion may also apply to other heart valves, vessels, and/or treatment locations within a patient with no or minimal changes to the structure and/or scope of the disclosure.

As seen in FIG. 2, a catheter 30 may be advanced through the patient's vasculature (e.g., the aorta 20, etc.) to and/or through the native heart valve (e.g., the aortic valve 12) into the ventricle 16 of the patient's heart 10. A distal end 32 of the catheter 30 may be disposed within the ventricle 16 of the patient's heart 10. The catheter 30 may include one or more lumens extending through the catheter 30 to the distal end 32. At least one of the one or more lumens may be a guidewire lumen and/or a working lumen. In some embodiments, the patient's heart 10 may be accessed more directly and navigation through the patient's vasculature (e.g., the aorta 20) may not be necessary.

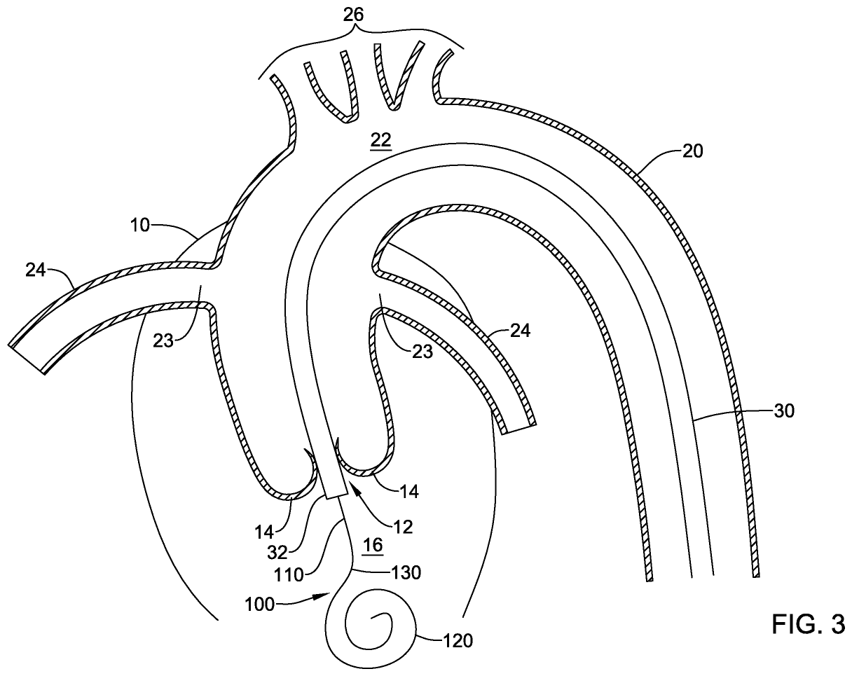

In some instances, the guidewire 100 may be advanced out the distal end 32 of the catheter 30 into the ventricle 16 of the patient's heart, as seen in FIG. 3. The guidewire 100 may be configured as described herein. In some instances, the guidewire 100 may be contained and/or constrained in a straightened configuration within the one or more lumens of the catheter 30 as the catheter 30 is advancing to the native heart valve (e.g., the aortic valve 12) of the patient's heart 10. In some instances, the guidewire 100 may be advanced simultaneously with and/or within the catheter 30 through the patient's vasculature to the native heart valve (e.g., the aortic valve 12) of the patient's heart 10. In some instances, the guidewire 100 may be advanced through the catheter 30 after positioning the distal end 32 of the catheter 30 within the ventricle 16 of the patient's heart 10.

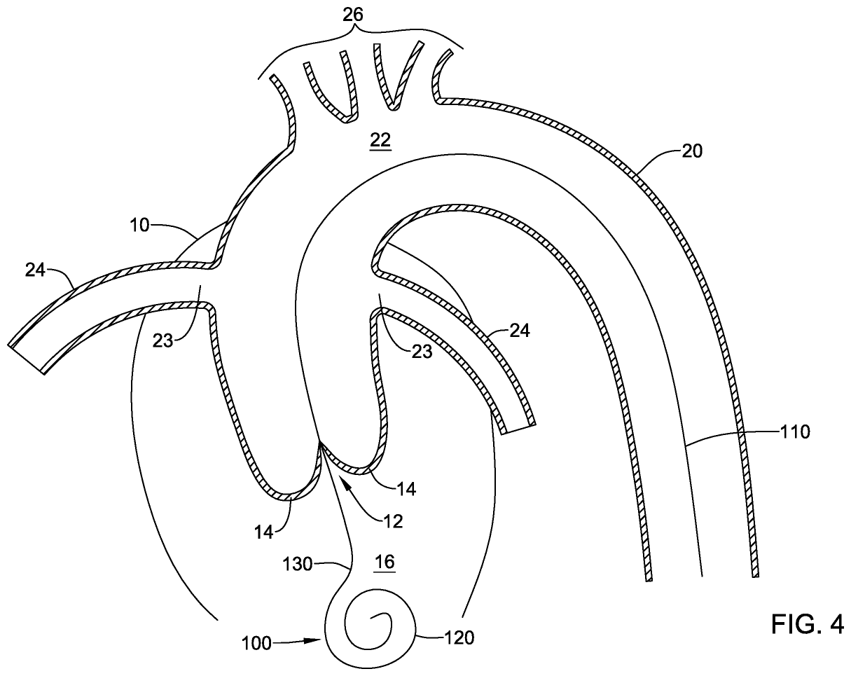

The coiled portion 120 of the guidewire 100 may be disposed within the ventricle 16 of the patient's heart 10 such that the distal portion of the proximal section 114 is spaced radially inward from an ostium of the native heart valve (e.g., the aortic valve 12) when the coiled portion 120 of the elongate shaft 110 of the guidewire 100 is positioned within the ventricle 16 of the patient's heart 10. The catheter 30 may be removed while maintaining the coiled portion 120 of the guidewire 100 within the ventricle 16 of the patient's heart 10, as shown in FIG. 4. FIG. 4 also illustrates the elongate shaft 110 spaced radially inwardly from the ostium of the native heart valve (e.g., the aortic valve 12) when the coiled portion 120 of the elongate shaft 110 of the guidewire 100 is positioned within the ventricle 16 of the patient's heart 10. The reverse curve portion 130 urges the distal portion of the proximal section 114 of the elongate shaft 110 toward a center of the native heart valve (e.g., the aortic valve 12).

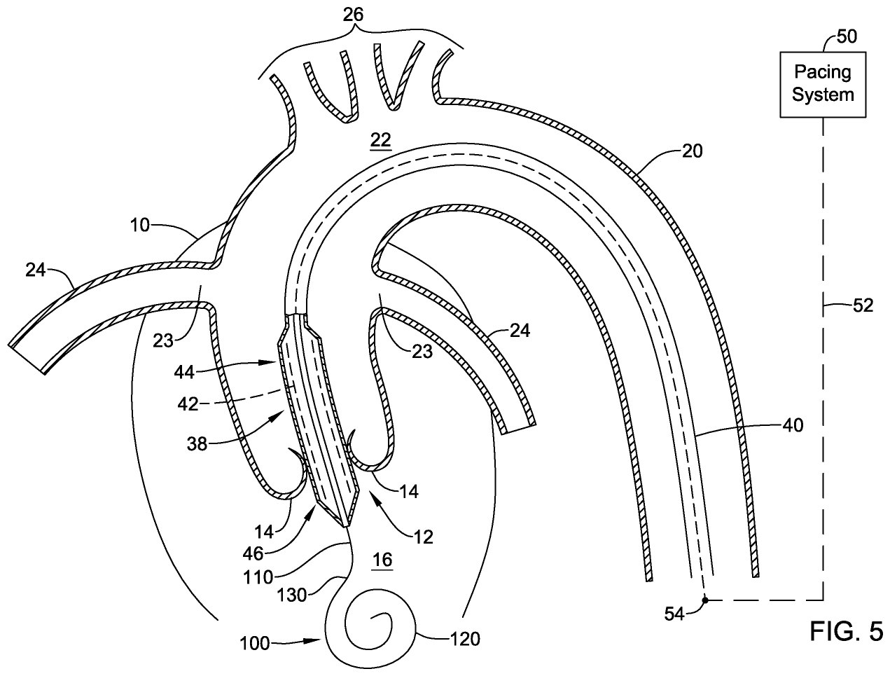

In some instances, a deployment device 40 may be advanced over the guidewire 100 to the native heart valve (e.g., the aortic valve 12) of the patient's heart 10, as seen in FIG. 5. The deployment device 40 is shown in a partial cutaway view. In some instances, the deployment device 40 may include an outer sheath and an inner shaft axially translatable relative to the outer sheath. In some instances, the inner shaft of the deployment device 40 may include a central guidewire lumen extending therethrough to a distal end of the deployment device 40.

The deployment device 40 and/or the outer sheath may include a distal containment section 38 having a replacement heart valve implant 42 disposed therein in a constrained and/or collapsed configuration. The distal containment section 38 may include a proximal portion 44 and a distal portion 46. In some instances, the proximal portion 44 and the distal portion 46 of the distal containment section 38 may be configured to axially translate relative to each other to open the distal containment section and release the replacement heart valve implant 42.

In some instances, the proximal portion 44 of the distal containment section 38 may be fixedly attached to and/or integrally formed with the outer sheath. In some instances, the distal portion 46 of the distal containment section 38 may be fixedly attached to and/or integrally formed with the inner shaft. Therefore, relative axial translation between the outer sheath and the inner shaft may cause corresponding relative axial translation of the proximal portion 44 of the distal containment section 38 and the distal portion 46 of the distal containment section 38 to open the distal containment section 38 and release the replacement heart valve implant 42. The distal containment section 38 of the deployment device 40 may be positioned adjacent to and/or within the native heart valve (e.g., the aortic valve 12) prior to opening the distal containment section 38 of the deployment device 40. In at least some instances, the distal portion 46 of the distal containment section 38 may be disposed at least partially upstream of the native heart valve (e.g., the aortic valve 12) and/or at least partially within the ventricle 16 of the patient's heart 10 prior to opening the distal containment section 38 of the deployment device 40.

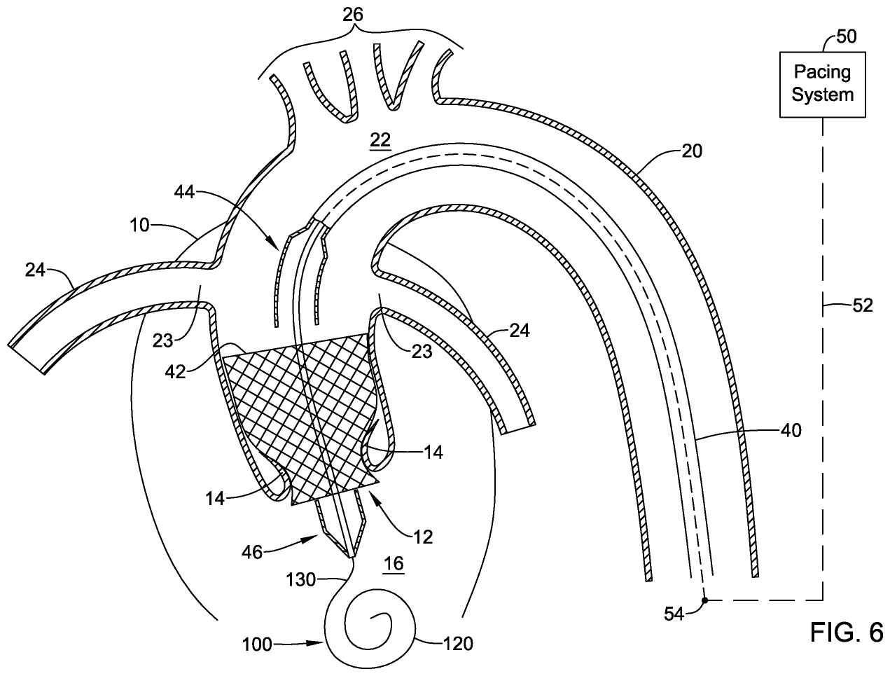

In some instances, the replacement heart valve implant 42 may be deployed within the native heart valve (e.g., the aortic valve 12) by opening the distal containment section 38 of the deployment device 40 while the distal containment section 38 is disposed within the native heart valve (e.g., the aortic valve 12), as seen in FIG. 6. The replacement heart valve implant 42 may include an expandable framework and a plurality of valve leaflets disposed within the expandable framework. In some instances, the expandable framework may be self-expanding. In some instances, the expandable framework may be mechanically and/or balloon expandable. Other configurations are also contemplated.

After opening the distal containment section, the distal portion 46 of the distal containment section 38 may be disposed upstream of the native heart valve (e.g., the aortic valve 12) and/or within the ventricle 16 of the patient's heart 10 and the proximal portion 44 of the distal containment section 38 may be disposed downstream of the native heart valve (e.g., the aortic valve 12) and/or within the aortic arch 22 and/or the aorta 20 of the patient. The reverse curve portion 130 of the elongate shaft 110 of the guidewire 100 may be at least partially disposed distal and/or upstream of the native heart valve (e.g., the aortic valve 12) and/or within the ventricle 16 of the patient's heart 10. Upon opening the distal containment section 38, the replacement heart valve implant 42 may shift toward and/or to an expanded deployed configuration. In some embodiments, the replacement heart valve implant 42 may be released from the distal containment section and/or the deployment device 40 prior to shifting to the expanded deployed configuration. In some instances, the replacement heart valve implant 42 may be released from the distal containment section and/or the deployment device 40 after shifting to the expanded deployed configuration.

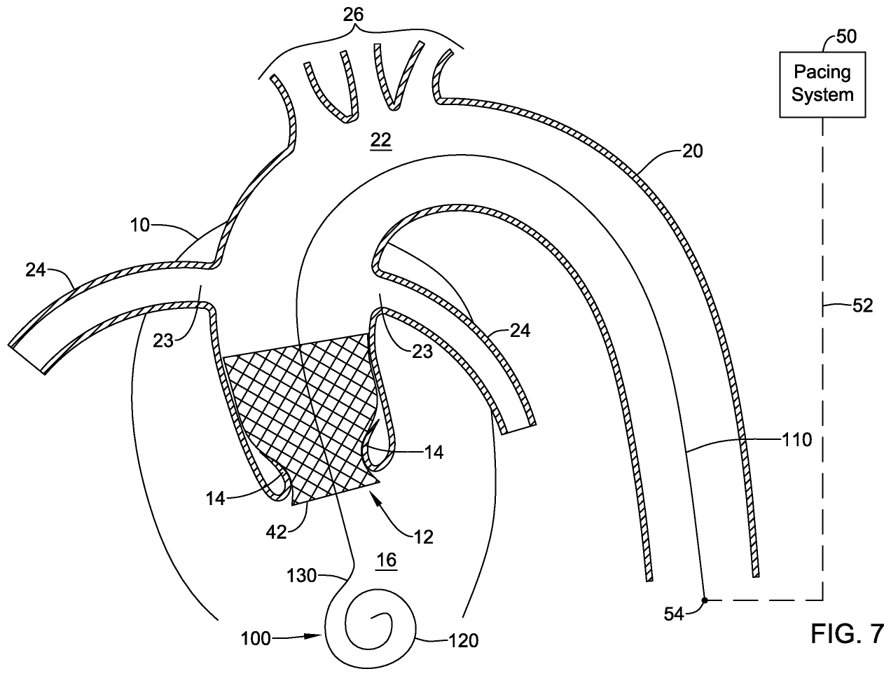

As can be seen in FIG. 6, the reverse curve portion 130 of the elongate shaft 110 of the guidewire 100 urges the distal portion of the proximal section 114 (not shown) of the elongate shaft 110 of the guidewire 100 toward a center of the native heart valve (e.g., the aortic valve 12) and/or away from the ostium of the native heart valve (e.g., the aortic valve 12). This positioning may prevent the distal portion 46 of the distal containment section 38 of the deployment device 40 from contacting the replacement heart valve implant 42 as the deployment device 40 is removed from the patient's heart 10, as seen in FIG. 7.

In some instances, the distal containment section 38 may be closed prior to removing the deployment device 40 from the patient's heart 10 and/or vasculature. In some instances, the deployment device 40 may be disposed within and/or may be retracted into a delivery catheter (not shown) prior to removal from the patient's heart 10 and/or vasculature. Other configurations are also contemplated.

In some instances, the catheter 30, the deployment device 40, the distal containment section 38, and/or elements thereof may include at least one radiopaque marker for visualization during delivery and/or navigation through the patient's vasculature. The at least one radiopaque marker may permit accurate placement under fluoroscopy of the distal end 32 of the catheter 30 and/or the distal containment section 38 of the deployment device 40 with respect to the native heart valve (e.g., the aortic valve 12), the ventricle 16, and/or the patient's heart 10.

In some instances, the physician or other professional implanting the replacement heart valve implant 42 may desire to use the guidewire 100 for delivering pacing pulses to the heart 10 during particular portions of the implantation process. In some instances, rapidly pacing the heart 10 during the implantation process may result in the heart 10 being less likely to displace the replacement heart valve implant 42 within the aortic valve 12. In some instances, as seen in FIGS. 5 through 7, a pacing system 50 may be utilized. The pacing system 50, which is shown schematically, may include a controller that determines when and how to pace, for example. The controller may also determine one or more pacing parameters for rapidly pacing the heart 10. An electrical cable 52 may extend from the pacing system 50, and may include a connector 54 that allows the electrical cable 52 to make an electrical connection with the guidewire 100. The connector 54 may take a variety of forms, depending on the construction of the proximal section 114 of the elongate shaft 110. The physician or other professional may utilize the pacing system 50 to pace during an appropriate step in implanting the replacement heart valve implant 42.

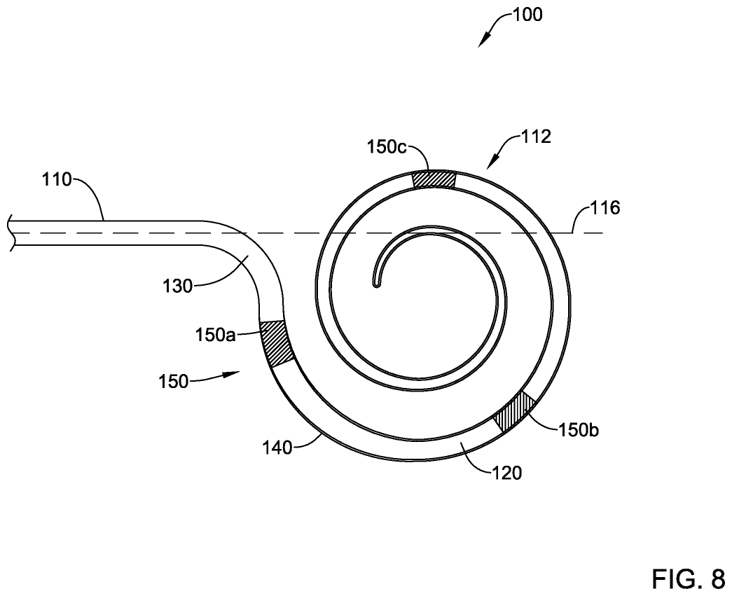

In some instances, the guidewire 100 may include one or more features that allow the guidewire 100 to be used for pacing the heart 10. In some instances, this may include one or more features that allow for the connector 54 to make repeatable electrical contact with the guidewire 100. In some instances, this may include one or more features that allow for electrical contact between cardiac tissue (such as within the ventricle 16 of the heart 10) and the coiled portion 120 of the guidewire. FIGS. 8 through 15 provide illustrative but non-limiting examples of some of these features.

FIG. 8 is a schematic view of the coiled portion 120 of the guidewire 100. In some instances, the coiled portion 120 includes one or more electrodes 150, individually labeled as 150a, 150b and 150c. While a total of three electrodes 150 are shown, it will be appreciated that this is merely illustrative, as the coiled portion 120 may include any number of electrodes 150. Because each patient is different, there are no guarantees that a single electrode 150, regardless of its placement on the coiled portion 120, may make contact with tissue within the ventricle 16. Accordingly, the coiled portion 120 may include a plurality of electrodes 150, each disposed at different positions on the coiled portion 120. In some instances, as shown, each of the electrodes 150 may represent a portion of the coiled portion 120 where the polymeric coating 140 has been removed to expose a metallic structure of the coiled portion 120. In some instances, the metallic structure of the coiled portion 120 may be formed of stainless steel or a nickel-titanium alloy such as nitinol. The metallic structure of the coiled portion 120 is electrically conductive.

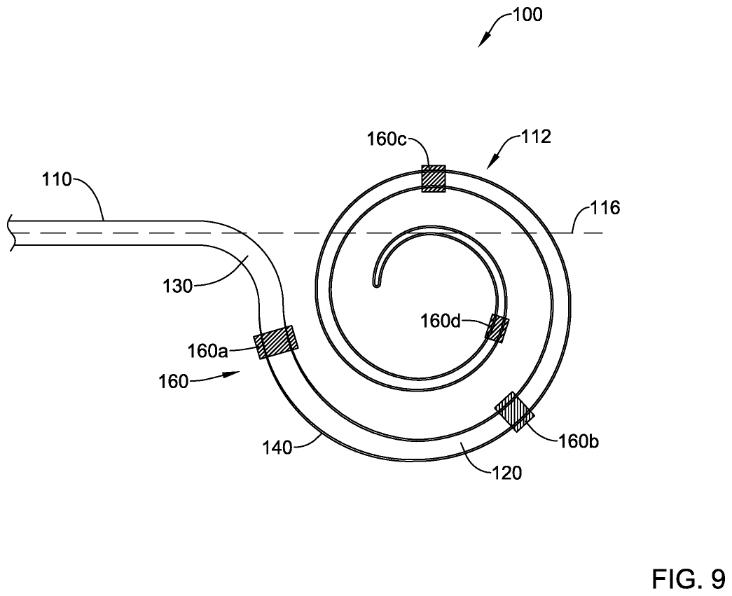

FIG. 9 is a schematic view of the coiled portion 120 of the guidewire 100. In some instances, the coiled portion 120 includes one or more electrodes 160, individually labeled as 160a, 160b, 160c and 160d. While a total of four electrodes 160 are shown, it will be appreciated that this is merely illustrative, as the coiled portion 120 may include any number of electrodes 160. Because each patient is different, there are no guarantees that a single electrode 160, regardless of its placement on the coiled portion 120, may make contact with tissue within the ventricle 16. Accordingly, the coiled portion 120 may include a plurality of electrodes 160, each disposed at different positions on the coiled portion 120. In some instances, as shown, each of the electrodes 160 may represent a conductive element that has been disposed on the coiled portion 120, and in electrical contact with the metallic structure of the coiled portion 120. The electrodes 160 may be formed of any suitable electrically conductive material.

FIGS. 8 and 9 provide several examples of how electrodes 150 and 160 may be incorporated in the coiled portion 120, and thus may be used to make electrical contact with tissue within the ventricle 16 in order to pace the heart 10 during the implantation process of the replacement heart valve implant 42. Being able to pace with the guidewire 100 also requires making good electrical contact with a portion of the guidewire 100 that extends outside of the patient's body, such as a proximal end of the proximal section 114 of the elongate shaft 110. It will be appreciated that the guidewire 100 has an electrically conductive core since the guidewire 100 is made of metal, along with perhaps insulative layers such as the polymeric coating 140 disposed over the electrically conductive core.

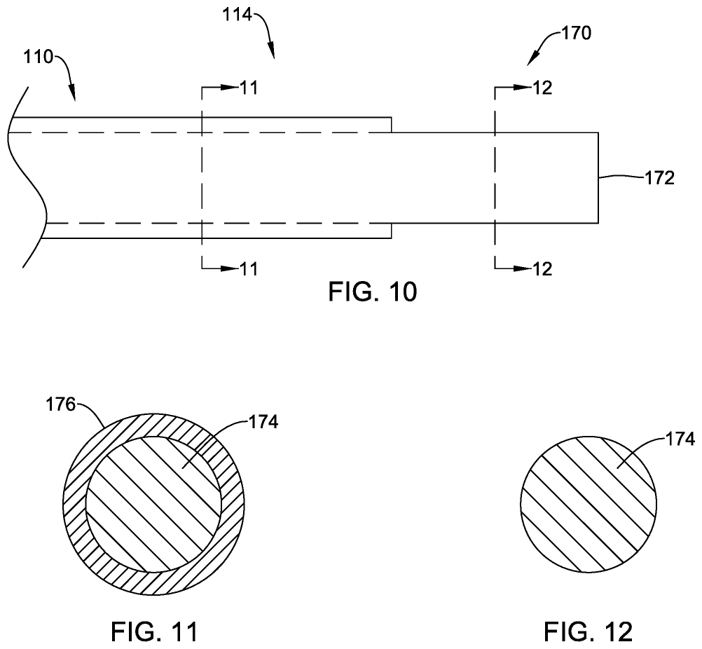

There are a variety of ways to provide the guidewire 100 with a proximal section 114 that is adapted to provide an electrical connection with the pacing system 50. In some instances, the proximal section 114 may include an electrical contact segment 170. The electrical contact segment 170 may be adapted to allow easy and repeatable electrical contact to be made between the connector 54 (FIGS. 5 to 7) and the electrical contact segment 170. FIG. 10 is a schematic view of the proximal section 114 of the elongate shaft 110. The electrical contact segment 170 extends distally a short distance from a proximal end 172 of the elongate shaft 110. In some instances, the proximal section 114 of the elongate shaft 110 may include a step-wise reduction in diameter moving proximally.

FIG. 11 is a cross-sectional view taken along the line 11-11 of FIG. 10 and FIG. 12 is a cross-sectional view taken along the line 12-12 of FIG. 10. FIG. 11 shows that the elongate shaft 110 includes a metallic core 174 that is encased in a polymeric covering 176. In some instances, the polymeric covering 176 extends distally and overlaps with the polymeric covering 140 over the coiled portion 120. Accordingly, essentially all of the elongate shaft 110 may be covered with an electrically insulating layer, with the exception of the electrodes 150 and 160 disposed within the coiled portion 120, and the electrical contact segment 170. In some instances, the electrical contact segment 170 represents a small portion of the proximal section 114 of the elongate shaft 110 in which the metallic core 174 is exposed.

As a result of the metallic core 174 being exposed within the electrical contact segment 170, a variety of different electrical connectors may be used as the connector 54 (FIGS. 5 to 7). In some instances, the connector 54 may simply be an alligator clip that can easily be clipped into position on the electrical contact segment 170 and can just as easily be removed once pacing is no longer desired. In some instances, the connector 54 may be a pacing tool such as the pacing tool described in commonly owned provisional patent application filed on the even date herewith, said provisional patent application having an Attorney Docket Number of 2001.3103100 and entitled PACING TOOL, which application is incorporated by reference herein.

FIG. 13 is a schematic view of the proximal section 114 of the elongate shaft 110. In some instances, as shown, the proximal section 114 may include an electrical contact segment 180. In some instances, the electrical contact segment 180 may include an electrically uninsulated guidewire core 182 having an insulated coil 184 placed over the uninsulated guidewire core 182. In some instances, the electrical contact segment 180 may also include a proximal end 186. In some instances, the proximal end 186 may be uninsulated and thus may be electrically conductive. The proximal end 186 may facilitate usage of the pacing tool described in the above-referenced provisional patent application that was incorporated by reference herein.

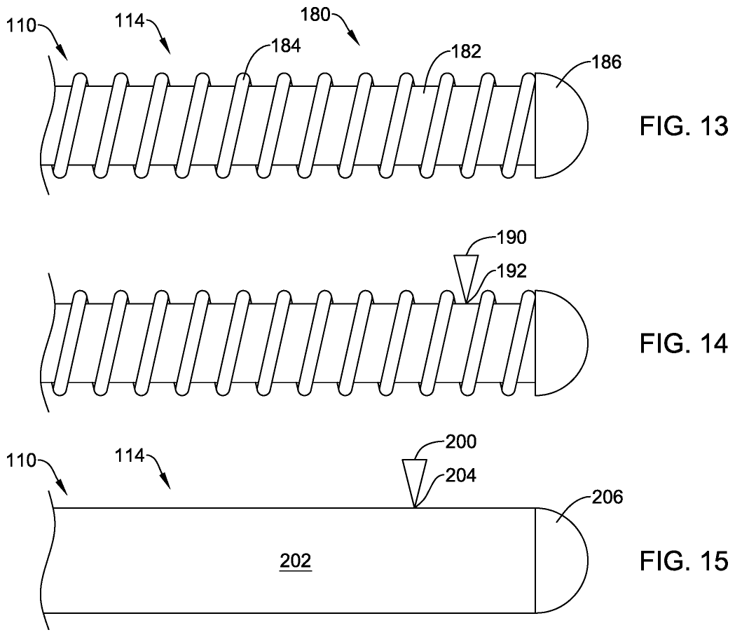

In some instances, as seen for example in FIG. 14, the electrical contact segment 180 may be adapted to be used in combination with a blade system 190. The blade system 190 may have a tapered yet blunt edge 192 that is dimensioned to fit between adjacent turnings of the insulated coil 184 in order to make electrical contact with the uninsulated guidewire core 182. In some instances, depending on the relative spacing between the adjacent turnings, the blade system 190 may be adapted to push the adjacent turnings farther apart in order to allow the blade system 190 to make electrical contact with the electrical contact segment 180. The blade system 190 may be considered as being an example of the connector 54 (FIGS. 5 through 7).

FIG. 15 is a schematic view of the proximal section 114 of the elongate shaft 110. In some instances, as shown, the proximal section 114 of the elongate shaft 110 may include an electrically insulating polymeric covering 202 that covers the core underneath. In some instances, a blade system 200 may have a sharp distal edge 204 that is adapted to cut through the electrically insulating polymeric covering 202 to make electrical contact with the core underneath. The blade system 200 may be considered as an example of the connector 54 (FIGS. 5 through 7). In some instances, the proximal section 114 of the elongate shaft 110 may include a proximal end 206. In some instances, the proximal end 206 may be uninsulated and thus may be electrically conductive. The proximal end 206 may facilitate usage of the pacing tool described in the above-referenced provisional patent application that was incorporated by reference herein.

The materials that can be used for the various components of the guidewire, the catheter, the replacement heart valve implant, the deployment device, and/or the forming tool (and/or other elements disclosed herein) and the various components thereof disclosed herein may include those commonly associated with medical devices. For simplicity purposes, the following discussion makes reference to the medical device(s). However, this is not intended to limit the devices and methods described herein, as the discussion may be applied to other elements, members, components, or devices disclosed herein, such as, but not limited to, the elongate shaft, the outer sheath, the inner shaft, etc. and/or elements or components thereof.

In some embodiments, the medical device(s) and/or other elements disclosed herein may be made from a metal, metal alloy, polymer (some examples of which are disclosed below), a metal-polymer composite, ceramics, combinations thereof, and the like, or other suitable material. Some examples of suitable metals and metal alloys include stainless steel, such as 444V, 444L, and 314LV stainless steel; mild steel; nickel-titanium alloy such as linear-clastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R44035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R44003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear than the super elastic plateau and/or flag region that may be seen with super elastic nitinol. Thus, for the purposes of this disclosure linear elastic and/or non-super-elastic nitinol may also be termed “substantially” linear elastic and/or non-super-clastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also be distinguished based on its composition), which may accept only about 0.2 to 0.44 percent strain before plastically deforming.

In some embodiments, the linear elastic and/or non-super-clastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (C) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-clastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear clastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of the medical device(s) and/or other elements disclosed herein may also be doped with, made of, or otherwise include a radiopaque material. Radiopaque materials are understood to be materials capable of producing a relatively bright image on a fluoroscopy screen or another imaging technique during a medical procedure. This relatively bright image aids a user in determining the location of the medical device(s) and/or other elements disclosed herein. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the medical device(s) and/or other elements disclosed herein to achieve the same result.

In some embodiments, a degree of Magnetic Resonance Imaging (MRI) compatibility is imparted into the medical device(s) and/or other elements disclosed herein. For example, the medical device(s) and/or components or portions thereof may be made of a material that does not substantially distort the image and create substantial artifacts (e.g., gaps in the image). Certain ferromagnetic materials, for example, may not be suitable because they may create artifacts in an MRI image. The medical device(s) or portions thereof, may also be made from a material that the MRI machine can image. Some materials that exhibit these characteristics include, for example, tungsten, cobalt-chromium-molybdenum alloys (e.g., UNS: R44003 such as ELGILOY®, PHYNOX®, and the like), nickel-cobalt-chromium-molybdenum alloys (e.g., UNS: R44035 such as MP35-N® and the like), nitinol, and the like, and others.

In some embodiments, the medical device(s) and/or other elements disclosed herein may be made from or include a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the medical device(s) and/or other elements disclosed herein may include a fabric material disposed over or within the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the medical device(s) and/or other elements disclosed herein may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum or a Ni—Co—Cr-based alloy. The yarns may further include carbon, glass or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the medical device(s) and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethylketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps, without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A guidewire for delivering a replacement heart valve implant, comprising:
an elongate shaft including a distal section and a proximal section extending proximally from the distal section;
a coiled portion disposed within the distal section; and
one or more electrodes disposed within the coiled portion, the one or more electrodes adapted for pacing the heart;
wherein the proximal section is adapted to provide an electrical connection with a pacing system.

2. The guidewire of claim 1, wherein the elongate shaft includes a polymeric coating disposed on the coiled portion.

3. The guidewire of claim 2, wherein the one or more electrodes disposed within the coiled portion comprise spots in the coiled portion in which the polymeric coating has been removed to expose metal underneath the polymeric coating.

4. The guidewire of claim 1, wherein at least some of the one or more electrodes are adapted to contact a ventricular wall of a heart when the coiled portion is disposed within a ventricle of the heart.

5. The guidewire of claim 1, wherein the proximal section of the elongate shaft includes a bare metal portion that is adapted to accommodate an alligator clip connecting the elongate shaft with the pacing system.

6. The guidewire of claim 1, wherein the proximal section of the elongate shaft includes a bare metal proximal end that is adapted to accommodate an electrical connection with the pacing system.

7. The guidewire of claim 1, wherein the elongate shaft includes a polymeric coating over an entirety of the elongate shaft with the exception of:
the one or more electrodes; and
a bare metal region of the proximal section.

8. The guidewire of claim 1, wherein at least a proximal-most region of the proximal section includes an uninsulated metal core and an insulated coil extending over the uninsulated metal core.

9. The guidewire of claim 1, wherein:
the coiled portion is curved in a first direction in a first plane;
the elongate shaft includes a reverse curve portion curved in a second direction opposite the first direction; and
the reverse curve portion is disposed proximal of the coiled portion.

10. The guidewire of claim 9, wherein the reverse curve portion is curved in the second direction within the first plane.

11. A guidewire adapted to deliver pacing pulses, comprising:
an elongate shaft including a distal section and a proximal section extending proximally from the distal section, the proximal section including an electrical contact segment adapted to make electrical contact with a pacing system;
wherein the elongate shaft is adapted for delivering a replacement heart valve implant, the elongate shaft including a coiled portion disposed within the distal section, the coiled portion curved in a first direction in a first plane, and wherein the elongate shaft includes a reverse curve portion curved in a second direction opposite the first direction;
wherein the elongate shaft includes a reverse curve portion curved in a second direction opposite the first direction, the reverse curved portion disposed proximal of the coiled portion; and
wherein the coiled portion includes one or more electrodes electrically coupled with the electrical contact segment.

12. The guidewire of claim 11, wherein the elongate shaft includes a polymeric coating disposed on the coiled portion.

13. The guidewire of claim 12, wherein the one or more electrodes electrically coupled with the electrical contact segment comprise spots in the coiled portion in which the polymeric coating has been removed to expose metal underneath the polymeric coating.

14. The guidewire of claim 11, wherein at least some of the one or more electrodes are adapted to contact a ventricular wall of a heart when the coiled portion is disposed within a ventricle of the heart.

15. The guidewire of claim 11, wherein the electrical contact segment comprises a bare metal portion that is adapted to accommodate an alligator clip connecting the elongate shaft with the pacing system.

16. The guidewire of claim 11, wherein the electrical contact segment comprises a bare metal proximal end that is adapted to accommodate an electrical connection with the pacing system.

17. The guidewire of claim 11, wherein the elongate shaft includes a polymeric coating over an entirety of the elongate shaft with the exception of:
the one or more electrodes; and
a bare metal region of the proximal section.

18. The guidewire of claim 11, wherein the electrical contact segment comprises an uninsulated metal core and an insulated coil extending over the uninsulated metal core.

19. A method of treating a native heart valve of a patient's heart, comprising:
advancing a catheter through the native heart valve into a ventricle of the patient's heart;
advancing a guidewire out a distal end of the catheter into the ventricle of the patient's heart, wherein the guidewire includes an elongate shaft including a distal section and a proximal section extending proximally from the distal section;
wherein the elongate shaft includes a coiled portion disposed in the distal section, the coiled portion being curved in a first direction in a first plane;
wherein the elongate shaft includes a reverse curve portion curved in a second direction opposite the first direction;
wherein the reverse curve portion is disposed proximal of the coiled portion;
positioning the coiled portion within the ventricle of the patient's heart such that a distal portion of the proximal section is spaced radially inward from an ostium of the native heart valve when the coiled portion of the elongate shaft is positioned within the ventricle of the patient's heart;
removing the catheter while maintaining the coiled portion of the elongate shaft within the ventricle of the patient's heart;
advancing a deployment device over the guidewire to the native heart valve;
rapidly pacing via the guidewire in order to temporarily interrupt native heart rhythm;
deploying a replacement heart valve implant within the native heart valve, the replacement heart valve implant including an expandable framework and a plurality of valve leaflets disposed within the expandable framework; and
withdrawing the deployment device without contacting the expandable framework of the replacement heart valve implant.

20. The method of claim 19, wherein the reverse curve portion urges the distal portion of the proximal section of the elongate shaft toward a center of the native heart valve.
